(19) United States
(12) Patent Application Publication
(10) Pub. No.: US 2017/0312253 A1
(43) Pub. Date: Nov. 2, 2017

(54) METHODS OF PREVENTING AND TREATING CEREBRAL AMYLOID ANGIOPATHY (CAA) WITH AN EDARAVONE MEDICAMENT

(71) Applicant: The Third Affiliated Hospital, Third Military Medical University, PLA, Chongqing (CN)

(72) Inventors: Yanjiang WANG, Chongqing (CN); Shusheng JIAO, Chongqing (CN); Xiuqing YAO, Chongqing (CN); Xinfu ZHOU, Adelaide (AU)

(21) Appl. No.: 15/523,855

(22) PCT Filed: Nov. 5, 2015
(86) PCT No.: PCT/CN2015/093837

§ 371 Date: May 2, 2017

(30) Foreign Application Priority Data
Nov. 5, 2014 (CN) 201410618300.6

(51) Int. Cl. A61K 31/4152; C07K 16/18; A61K 39/395

(52) U.S. Cl. CPC A61K 31/4152; A61K 39/3955; C07K 16/18

(57) ABSTRACT

The present invention belongs to pharmaceutical field, specifically, relates to use of Edaravone in the preparation of a medicament for preventing and treating cerebral amyloid angiopathy (CAA). By administering Edaravone, the present invention could scavenge the deposited amyloid (Aβ) in the brain and prevent the intracerebral Aβ deposition.

### FIELD OF THE INVENTION

The present invention belongs to the pharmaceutical field. Specifically, the present application relates to the use of Edaravone for preventing and scavenging intracerebral AP deposition, in particular for preventing and treating cerebral amyloid angiopathy (CAA).

### BACKGROUND

Cerebral amyloid angiopathy (CAA) refers to the deposition of β-amyloid (Aβ) on the media and adventitia of small and mid-sized arteries (and, less frequently, veins) of the cerebral cortex and the medulla. It is a component of any disorder in which amyloid is deposited in the brain. CAA has been recognized as one of the morphologic hallmarks of Alzheimer disease (AD), bust it is also often found in the brains of elderly patients who are neurologically healthy. While often asymptomatic, CAA may lead to intracranial hemorrhage (ICH), dementia or transient neurologic events, in which ICH is the most recognized result. Effective therapeutics for CAA is currently not available.

Edaravone, chemical structure being 3-methyl-1-phenyl-2-pyrazolin-5-one and molecular Formula being C₁₀H₁₀N₂O, contains three antioxidant groups (see, for example, Edaravone (3-methyl-1-phenyl-2-pyrazolin5-one), a novel free radical scavenger, for treatment of cardiovascular diseases, Higashi Y I, Jitsuiki D, Chayama K, Yoshizurai M., Recent Pat Cardiovasc Drug Discov., Jan. 2006; 1(1):85-93, and The reaction rate of edaravone (3-methyl4-phenyl-2pyrazolin-5-one (MCI-186)) with hydroxyl radical, Abe S, Kirima K, Tsuchiya K, Okamoto M, Hasegawa T, Houchi H, Yoshizumi M, Tamaki T. Chem Pharm Bull (Tokyo), Feb. 2004; 52(2):186-91), which has potent antioxidant effects and radical scavenging effects. It is a medicine currently approved for treating acute ischemic stroke in clinical practice, which exerts neuroprotective effects by scavenging the oxygen free radicals generated during the ischemia-reperfusion. It is found in the previous studies that Edaravone could reduce cerebral ischemia-reperfusion injury area, and suppress the gene expression of the Fas/FasL signaling pathway and thereby inhibit neuronal apoptosis (see, for example, Edaravone neuroprotection effected by suppressing the gene expression of the Fas signal pathway following transient focal ischemia in rats, Xiao B, Bi F F, Hu Y Q, Tian F F, Wu Z G, Mujlli H M, Ding L, Zhou XF, Neurotox Res., Oct, 2007; 12(3):155-62). It is found in a recent research that Edaravone could inhibit the mitochondrion dependant apoptosis pathway in the N2a/Swe.Δ9 cells. In the ischemia-reperfused elderly rats, Edaravone could reduce the glutathione peroxidase activity, suppress the JNK-c-Jun pathway and decrease the neuronal apoptosis. Edaravone has therapeutic effects on other nervous system degenerative diseases such as amyotrophic lateral sclerosis (ALS) and Parkinson's disease.

### SUMMARY OF THE INVENTION

It is firstly discovered by the present inventors that Edaravone directly acts on Aβ, inhibits Aβ aggregation, promotes Aβ fiber disaggregation, influences Aβ metabolism, thereby blocks or delays the progression of cerebral amyloid angiopathy (CAA).

The present invention provides a method for treating CAA, comprising administering a therapeutically effective amount of Edaravone or its analogue or derivative thereof to a patient suffering from or suspected of suffering from CAA.

According to the present invention, Edaravone is administered intravenously, subcutaneously or orally, preferably administered orally.

Edaravone can be formulated into an injection, a tablet, a capsule, etc.

Edaravone can be administered in combination with other CAA therapeutic medicine, such as an Aβ antibody, if desired.

According to the present invention, the method further comprises determining that the patient suffers from CAA, wherein the determination step occurs before the administration step. The determination step is for determining the clinical symptoms of the patient suffering from CAA.

According to the present invention, the patient has or does not have characteristic plaques of Alzheimer's disease in the brain.

According to the present invention, the patient has or does not have symptoms of Alzheimer's disease.

According to the present invention, the patient has experienced or has not experienced a heart attack or stroke.

According to the present invention, the daily dose of Edaravone is between about 0.1 mg/kg and about 25 mg/kg. The daily dose is preferably 0.5-5.0 mg/kg for prevention purpose, while preferably 5.0-25 mg/kg for treatment purpose. For example, the daily dose is about 0.5 mg/kg, 3.5 mg/kg or 15 mg/kg, bid or biw.

According to the present invention, the method further comprises monitoring the change of the signs or symptoms corresponding to the CAA during administration period.

The present invention further provides a method for preventing CAA, comprising administering an effective amount of Edaravone or its analogue or derivative thereof to a patient susceptible to CAA.

The present invention further provides a method for reducing a vascular amyloid protein of a patient, comprising administering Edaravone according to a therapeutic regime that is related to the removal of the vascular amyloid protein and reduces the incidence of cerebral microbleeds. In one embodiment, the method further comprises monitoring the cerebral microbleeds of the patient by MRI. In another embodiment, the method further comprises monitoring the removal of the vascular amyloid protein of the patient by a PET scan. The therapeutic regime can be a long-term therapeutic regime.

### Edaravone and Analogues or Derivatives Thereof

The Edaravone has the chemical name of 3-methyl-1-pheneyl-2-pyrazolin-5-one, molecular formula of C₁₀H₁₀N₂O, molecular weight of 174.19, and the chemical structural formula of

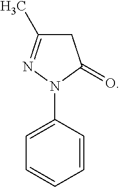

Analogues of Edaravone comprise those in which methyl at the position 3 of the pyrazolone ring may be replaced with a lower (C₁₋₆) alkyl such as ethyl, propyl, etc., or replaced with a lower alkoxy such as methoxy, ethoxv, etc., or methyl at position 3 may be replaced with H while the position 4 is substituted with a lower alkyl or alkoxy. Derivatives of Edaravone comprise esters, i.e., those in which ketone in position 5 of the pyrazoline ring is transformed into enol, and reacts with a carboxylic acid to generate esters such as methyl esters, ethyl esters, etc. The ester (precursor) is converted again into ketone after hydrolysis in vivo. In addition, phenyl is also optionally substituted with one or more substituents that are selected from a lower alkyl, a lower alkoxy, nitro, halogen, etc.

It is expected in the present invention that the above analogues and derivatives have the equivalent function with Edaravone.

It is reported that a hereditary factor plays a role in certain types of CAA and CAA-related diseases. Therefore, it is optionally to determine the symptom, sign or the presence or absence of the risk factors of the disease before treatment.

### Diagnosis and the Monitoring of a CAA Patient

Like most neurologic diseases, diagnosis often comes up on the basis of a medicai history of the patient, a careful inquiry of the family history, onset eases of the patient, a symptomatic pattern, as well as a neurological examination. Brain computerized tomoscanning (CT) or magnetic resonance imaging (MRI) may identify lobar hemorrhage, stroke or petechial hemorrhage, which are very important in excluding arteriovenous malformation, brain turner or other hemorrhage causes. Angiography (an X-ray test of the inside of blood vessel and heart) is no help for the diagnosis of CAA, but it may be desired for excluding aneurysm. Brain biopsy (excision of small pieces of brain tissue) could show a characteristic amyloid deposit. When the diagnosis is uncertain, biopsy may be needed to exclude potential diseases to be treated. Lumbar puncture that determines a cerebrospinal fluid protein could show a characteristic abnormality.

CAA accompanied by hemorrhage must be distinguished from the other types of cerebral hemorrhage. In CAA, hemorrhage usually occurs in the lobe, the blood, after rupture, often enters the subarachnoid space between brain and the capsule during the night. In hypertension correlated hemorrhage, hemorrhage often occurs deeper in the brain, the blood, after rupture, enters the chamber or cavity deep in the brain during the daytime activities. Other causes for brain hemorrhage include arteriovenous malformation, wound, aneurysm, bleeding in brain tumors, vasculitis (vascular inflammation) or hemorrhagic disorders. Cerebral microbleeds of the patient may be monitored by MRI and/or the removal of the vascular amyloid protein of the patient may be monitored by a positron emission tomography (PET) scan.

Patients suitable for treatment include the individuals that are under the risk of CAA while no symptom is shown, as well as the patients who are currently showing the symptoms.

### Therapeutic Regimes

In the prophylactic use, the regime for administering a pharmaceutical composition or agent to the patient susceptible to CAA or under the risk of CAA comprises administering the composition or agent with an amount and frequency sufficient to eliminate or reduce the risk, mitigate the severity or delay the onset of disease, including the physiological, biochemical, historical and/or behavioral symptoms of the disease, complications and intermediate pathological phenotypes shown during the development of the disease. In the therapeutic use, the regime for administering a pharmaceutical composition or agent to the patient having or being suspected of having such disease comprises administering the composition with an amount and frequency sufficient to cure or at least partially suppress the symptom of the disease, including the complications and intermediate pathological phenotypes shown during the development of the disease. The appropriate amount to complete the therapeutic or prophylactic therapy is defined as a therapeutically or prophylactically effective amount. The combination of the appropriate amount and administration frequency to complete therapeutic or prophylactic therapy is defined as a therapeutically or prophylactically effective regime.

The administration amount and frequency of the present agent may depend on the prophylactic or therapeutic application of the therapy. In the prophylactic application, a pharmaceutical composition comprising Edaravone is administered to the patient who is not yet at a state of disease to enhance his/her resistivity. This amount is defined as “a prophylactically effective amount”. In this application, the precise amount still depends on the health status of the patient, but is usually at 0.1-25 mg per dose, especially in the range of 0.5-5.0 mg per dose. A relatively low amount is administered at a relatively infrequent interval over a long period of time. Some patients receive the permanent treatment in the rest of their lives.

In the therapeutic application, it may sometimes requires administering a relatively higher amount (for example, about 3.5-25 mg per dose, or 5.0-25 mg per dose) at a relatively shorter interval until the development of the disease mitigates or terminates, preferably until partial or complete improvement of the symptom is shown in the patient. After that, a prophylactic scheme may be applied to the patient.

The presently claimed pharmaceutical agent may optionally be administered in combination with other agents that are at least partially effective in the treatment of CAA or AD. In the case of CAA when an amyloid deposit is present in the cerebrovascular system, the presently claimed pharmaceutical agent may also be used in combination with other drugs that could enhance the migration of the presently claimed agent across the blood-brain barrier.

The presently claimed agent may be administered parenterally, topically, intravenously, orally, subcutaneously, intraarterially, intracranially, intrathecally, intraperitoneally, intranasally or intramuscularly for the prophylactic and/or therapeutic treatment. Although other routes can be equally effective, a typical route of administration is an oral administration, secondly an intramuscular injection. This kind of injection is most frequently carried out in the muscles of arms or legs. In some methods, the agent is directly injected into the specific tissues where the deposit accumulates, for example, by intracranial injection. Intramuscular injection or intravenous infusion is also preferred. In some methods, a specific therapeutic antibody is directly injected intracranially.

The beneficial effect of the present invention are mainly embodied in that Edaravone or the analogues or derivatives thereof have an effect of inhibiting Aβ aggregation, thereby Aβ that already deposited in the intracerebral vascular wall or in the cerebral parenchyma can be eliminated, or a continuous deposition of Aβ in the intracerebral vascular wall and the cerabral parenchyma can be prevented.

### BRIEF DESCRIPTION OF THE DRAWINGS

For more clearly describing the technical solution of the present invention, brief introduction will be made in conjunction with the accompanying drawings. Obviously, these drawings are merely some specific embodiments recorded in the present application. The technical solutions of the present invention include but not limited to these drawings.

FIG. 1 illustrates that Edaravone has a significant aggregation-inhibitive and disaggregatiye effect on Aβ. Wherein, 1: the molecular structure of Edaravone; 2 and 3: a Thioflavin T test demonstrates that Edaravone has a significant aggregation-inhibitive and disaggregatiye effecton on Aβ; 4-6; a Western blot farther demonstrates that Edaravone has a significant aggregation-inhibitive effect on Aβ; 7-10: a transmission electron microscope further confirms that Edaravone has a significant aggregation-inhibitive and disaggregative effect on Aβ.

FIG. 2 illustrates that Edaravone could antagonize the toxic effect of Aβ on cells. Wherein, 1-3: Edaravone could antagonize the toxic effect of Aβ on SH-SY5Y cells, wherein the cell survival rate and the cellular neurite length are both higher than the simple Aβ treated group; 4-5: Edaravone could antagonize the toxic effect of Aβ on primary cultured cortical neurons, wherein the neurite lengths of the cortical neuron in the Edaravone treated group are significantly higher than in the simple Aβ treated group: 6-7: Edaravone significantly reduces the Aβ induced apopiosis; 8: a ROS test demonstrates that Edaravone significantly reduces the intracellular oxidation level.

FIG. 3 illustrates that Edaravone significantly improves the intelligent level of AD mice. Wherein, 1-2: a Morris water maze demonstrates that the escape latency and the times across the platform of the mice in the Edaravone prevention group are significantly lower than those in the control group; 3 and 4: the mice in the Edaravone prevention group perform much better in a Y-maze test than in the control group; 5-7: the mice in the Edaravone prevention group perform much better in an open field test than in the control group; 8-10: the mice in the Edaravone treatment group perform much better in the Morris water maze than in the control group.

FIG. 4 illustrates that Edaravone significantly reduces the Aβ deposition in the brain and in the blood vessels in the brain. Wherein 1-2: in the Edaravone prophylactic test, Edaravone significantly lowers the Aβ deposition in the brain; 4-5; in the Edaravone therapeutic test, Edaravone significantly lowers the Aβ deposition in the brain; 3 and 6: regardless in the prophylactic, or therapeutic test, Edaravone could significantly reduce the Aβ level in the brain; 7-8: regardless in the prophylactic or therapeutic test, Edaravone could significantly reduce the Aβ deposition in the blood vessels in the brain.

FIG. 5 illustrates that Edaravone inhibits the BACE and Gsk-3β activities. Wherein: 1-5: Edaravone significantly reduces the CTF β and Aβ expressions in the brain of the AD mice; 6-7: Edaravone inhibits the B ACE expressions and activities in the brain of the AD mice, and increases the α-secretase activities in the brain of the AD mice; 8: Edaravone inhibits the Gsk-3β activities in the brain of the AD mice; 9-10: an in vitro test further demonstrates that Edaravone inhibits the BACE expression; 11: in vitro test further demonstrates that Edaravone inhibits the Gsk-3β activities.

FIG. 6 illustrates that Edaravone significantly improves the Aβ-induced secondary pathological changes. Wherein, 1-3: Edaravone could significantly reduce the damage to the structures of the neurons and the neuron losses in the brain of the AD mice; 4: Edaravone could significantly reduce the neuron apoptosis in the brain of the AD mice; 3: Edaravone could significantly reduce the reactivities of astrocytes and microglias in the brain of the AD mice; 6-7: Edaravone could significantly the phosphorylation degree of Tau proteins in the brain of the AD mice; 8-9: Edaravone could protect the integrity of the synapse structures in the brain of the AD mice; 10: Edaravone could significantly reduce the neuroinflammation in the brain of the AD mice.

FIG. 7 illustrates that Edaravone significantly reduces the oxidation level in the brain of the AD mice.

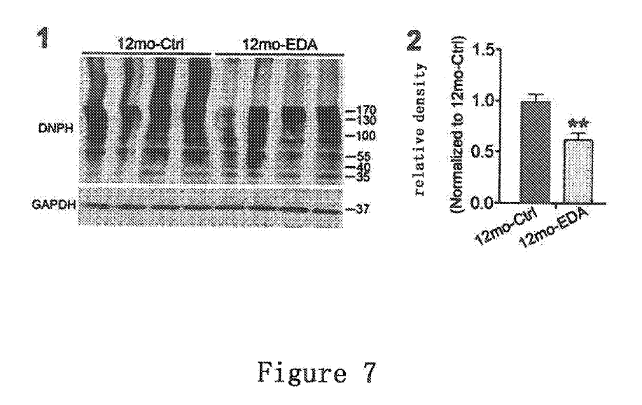

### DETAILED DESCRIPTION OF THE INVENTION

For further understanding the present invention, the preferred technical solutions of the present invention will be described in association with examples below. These descriptions only illustrate the features and the advantages of the technical solutions of the present invention and do not limit the scope of the present invention.

### 1. A Thioflavin T Fluorescence Test

An inhibitive effect of Edaravone on Aβ aggregation: solutions of 10 μM Aβ 42 and different concentrations of Edaravone (0 μM, 1.56 μM, 3.13 μM, 6,25 μM, 11.25 μM, 25.0 μM, 50.0 μM and 100 μM) were incubated in an incubator at 37° C. for two days, a 5 μM thioflavin T working solution was added thereto and continued the incubation for 20 min, and optical density (OD) values were obtained under a microplate reader (excitation wavelength: 450 nm, emission wavelength: 482 nm).

A disaggregative effect of Edaravone on Aβ fibers: Aβ 42 was dissolved in the DMEM cultures and incubated at 37° C. for two days to form Aβ fibers. The solutions of the formed Aβ fibers and different concentrations of Edaravone (concrete concentrations are the same as above) were incubated under the same conditions for another three days, a 5 μM thioflavin T working solution was added and incubated for 20 min, OD values were obtained under the microplate reader (excitation wavelength and emission wavelength are the same as above).

### 2. A Transmission Electron Microscope Negative Staining

A transmission electron microscope (TEM) negative staining was applied in the present research to further confirm the inhibitive effect on Aβ fiber formation and the disaggregative effect on the formed Aβ fibers of the Edaravone. Formvar coated copper grid was put on the bottom of a 24-well plate beforehand, and different concentrations of Edaravone working solutions were incubated together with Aβ in the 24-well plate. The treatment of Aβ and the subsequent incubation process are the same as in the thioflavin T test. After that, superfluous liquid was removed, stained using phosphotungstic acid for 30 s. The samples were naturally air-dried, observed and taken pictures by Joel 1200 EX transmission electron microscope.

### 3. A Western Blot Determination of the Aggregation-Inhibitive Effect of Edaravone on Aβ

Solutions of 1 μM Aβ42 and different concentrations of Edaravone (0 μM, 0.3 μM, 1 μM and 3 μM) were incubated in a incubator at 37° C. for two days, the intermixture and a non-reductive loading buffer were mixed gently, Western blot was carried out with the detection antibody being a Aβ antibody (6E10) derived from a mouse.

### 4. A SH-SY5Y Cell Line Culture, Cell Viability Assay and Neurite Growth Detection

The SH-SY5Y cell line was purchased from the Peking Union Hospital Cell Bank, cultured in a humidified incubator under 5% CO and 37° C., A MTT cell viability assay was performed according to the instructions in the kit (Sigma). The SH-SY5Y cells were treated with mixed working solutions of Aβ and different concentrations of Edaravone for 24 h, then were incubated with a MTT working solution (0.5 mg/ml) at 37° C., then incubated with 10% SDS for 15 min. The optical density values were determined at a wavelength of 563 nm (Synergy H4, Bio Tek). The neurite growth test was performed according to the method described in the prior documents (see, for example, Wang, Y. J., et al., Effects of proNGF on neuronal viability, neurite growth and amyloid-beta metabolism. Neurotox Res, 2010, 17(3): p. 257-67). The SH-SY5Y cells were cultured in the medium containing 1% FBS and 10 μM all-trans retinoic acid (RA) (Sigma, US) for 7 days, and the cells were treated with 1 μM Aβ and different concentrations of Edaravone for 24 h, observed under the inverted microscope, taken pictures, and the neurite lengths of the 50 neurons per group were measured using ImageJ software.

5. A Primary Culture of Cortical Neurons, a ROS Detection, and Labeling Apoptotic Cells with Propidium Iodide (PI)

Cortical neurons were separated from the brains of the new born 129sv mice., and cultured on the poly-L-lysine coated cover glasses. 72 h later, the cells were treated with working solutions of 1 μM Aβ 42 oligomers and different concentrations of Edaravone for 24 h, then fixed with 4% paraform for 30 min, then stained with MAP-2 antibodies. The method for neurite length measurement is as mentioned above.

The ROS detection was performed according to the scheme provided by the manufacturer (Cell Biolabs Inc).

The method for labelling the apoptotic neurons with propidium iodide (PI) are as follows: living neurons were washed with a PBS solution for three times; the washed living neurons were incubated with a PI working solution (2 μg/ml) at room temperature for 15 min, and then washed with a HEPES buffer solution (100 mM HEPES, 140 mM NaCl, 25 mM CaCl₂, pH 7.4), 5 min×3. The cells were finally fixed with 2% paraform in PBS for 20 min, then washed with buffer solution, 5 min×3, and incubated with DAPI (1:1000) 20 min for counterstaining. The cells were observed and taken pictures under B50 fluorescence microscope.

### 6. Animals and Edaravone Administration

APPswe/PS1DE9 (AD) transgenic mice were purchased from Jackson Laboratory, USA (No. 005864), raised in the SPF grade animal house of The Third Affiliated Hospital of The Third Military Medical University. All the processes and correlated experiments applied to the mice were approved and filed with the Animal Ethics Committee of the Third Military Medical University. In the prophylactic test, 3-month old AD mice received Edaravone treatment for six months, the blank control (AD mice) and normal wild type control (wild type litter mate) received isometrical normal saline, in the therapeutic test, 9-month old AD mice received Edaravone treatment for three months, blank control (AD mice) and normal wild type control at the same age were also set up. The equivalent therapeutic dose of Edaravone in mice was deduced from the dose for adults: Edaravone injection 12.6 mg/kg, ip, twice a week. Meanwhile, the mice of the other two groups were administered with Edaravone orally, with a daily dose of 0.1-25 mg/kg, for example, 3.5 mg/kg (both suitable for the prophylaxis and therapy purpose). Alternatively, the daily dose for the prophylactic group may be 6.4 mg/kg, and the daily dose for the treatment group may be 12.8 mg/kg. Administration from 3-month old to 9-month old is the prophylactic test, and administration from 9-month old to 12-month old is the therapeutic test After reaching a stated time, the mice in each group received behavioral tests according to the method in the follows. Three mice in each group were taken for Golgi staining. The remaining mice were overly anaesthetized to receive brain anatomy. One side of the cerebral hemisphere was frozen and then for a biochemical analysis, and the other side of the cerebral hemisphere was fixed with 4% paraform for a histological analysis.

### 7. Behavioral Tests

All the mice in each group received behavioral tests, including a water maze, Y-maze and open field test. Water maze test includes four days of platform test and the subsequent probe test. The entire process was recorded by video, and analyzed with an image analysis software (ANY-maze, Stoelting). In the platform test, the escape path length and escape latency were measured and calculated. In the probe test, the time in each quadrant and the time across platform were measured. The spontaneous alternation test and novel arm probe test were carried out in the Y-maze. In the spontaneous alternation test, the mouse was free running In the maze for 5 min. Alternation is defined as entering the three arms connected with each other continuously. The percentage of alternation is the ratio of actual number of alternation to the maximum number of possible alternation (defined as the arm entry minus 2) multiplied by 100. In the novel arm probe test, one arm (novel arm) is blocked, the mouse was allowed to probe in the other two arms for 5 min. After a two-hour intermission, the mouse may probe the three arms freely. In the open field test, the mouse was put in the center of an open field for 3 min, and its path was recorded by a tracking system (Limelight, ActiMetrics). At the same time, the numbers of rearing, grooming, defecation and urination of the mouse were recorded.

### 8. A Quantitative Analysis of the Aβ Deposition in the Brain

The compact Aβ plaque was stained and labelled with Congo red. A equidistant (˜1.3 mm) frozen section was treated with a sodium chloride working solution at room temperature for 20 min, with a Congo red working solution treated for 45 min, and rapidly dehydrated with absolute alcohol. The total Aβ plaques were labelled with 6E10 immunohistochemistry. 6E10 staining was performed by a method of bleach section. The Aβ plaques in the neopallium and hippocampal area were quantitatively analyzed with ImageJ analysis software to obtain the area percentage of the Aβ plaque and the quantities in a unit area.

### 9. A Cerebrovascular Amyloidosis Detection

A new assessment method was applied in the present research to assess the severity of the cerebrovascular amyloidosis. The blood vessels in the brain were double immunofluorescence stained with α-smooth muscle action antibody (1a4) and Aβ antibody (6E10), observed under eonfocal laser microscopy (Radiance 2000MP, Bio-Rad), and the images of the blood vessels were recorded. The severity of the CAA of each blood vessel was scored based on the following four-level scale: level 0: no Aβ deposition; level 1: little Aβ deposition, Aβ deposition occupies less than one third of the perimeter of the blood vessel; level 2: moderate Aβ deposition, Aβ deposition occupies more than one third but less than two thirds of the perimeter of the blood vessel; level 3: severe deposition, Aβ deposition occupies more than 75% of the perimeter of the blood vessel.

### 10. Staining and Quantitative Analysis of Microbleeds

First, 2% potassium ferrocyanide was used to stain the hemosiderin, then 1% Nuclear Fast Red was used to counterstain the cell nucleus. The microbleeds in each brain tissue slice were quantified under microscopy, and the average of the hemosiderin precipitations contained in each slice was calculated.

### 11. ELISA Analysis

After homogenating the frozen brain tissues, extraction was conducted with TBS, SDS and formic acid successively. The TBS, SDS and formic acid extracts represent soluble Aβ, loosen senile plaques and compact senile plaques, respectively. An ELISA kit (Covance or eBioscience) was used to determine the concentrations of Aβ 40, Aβ 42 (Covance), IL-6, IL-1β, INF-γ, TNF-β (eBioscience, BMS603, BMS6002, BMS606, BMS607) in the brain and serum. The determination steps were carried out according to the instructions of the kit. The determinations of the activities of alpha and beta secretases in the brain tissues were also carried out according to the instructions of the kit (R&D Systems).

### 12. Immunostaining of Neuron NeuN, ChAT and MAP-2

To clarify the protective effect of Edaravone on neurons, immunohistochemistry detections of NeuN, ChAT and MAP-2 in the method of bleach section were carried out to the brain tissue slices. Primary antibodies were rabbit-anti-mouse NeuN, ChAT and MAP-2antibodies, respectively, with a dilution ratio of 1:200, incubated at 4° C. overnight, biotinylated Ig secondary antibodies were incubated at 37° C. for 2 h, followed by DAB colouration, serial dehydration, transparentized and mounting.

### 13. Astrocyte Reaction, Microglia Reaction and Tau Hyperphosphorylation Immunostaining

Immunohistochemistry in the method of bleach section was carried out according to the previous steps. The primary antibodies were rabbit-anti-mouse CD45 antibody (for labelling microglias), rabbit-anti-mouse GFAP (for labelling astrocytes) and rabbit-anti-mouse pSer396-Tau antibody, respectively. The dilution ratio was 1:200, the following steps were the same as above.

### 14. Golgi Staining

After overly anesthetized, the animals received heart perfusions with normal saline containing 0.5% nitrous acid, 4% paraform, and Golgi dye liquor, successively. After perfusion, the brain tissues were cut into 0.1 cm×0.1 cm pieces, transferred to brown vessels containing Golgi dye liquor, left without light at room temperature for 3 d, and then immersed in the 1% silver nitrate solution for further incubation, reacted without light for 3 d. The brain tissues were sliced with vibratome (Leica, VT1000 S, Germany) at a slice thickness of 35 μm. The numbers of the dendritic spines within 10 μm of the central dendrites of the pyramidal cells in the hippocampus CA1 region were accounted.

15. Determination of an Oxidation Level in the Brain Tissues with Dinitrophenylhydrazine (DNPH) Western Blotting

The carbonyl level of the proteins in the tissue reflects the oxidation level in the tissue. DNPH is reacted with the carbonyl of the proteins to form a protein-DNP hydrazone complex, and the level of the complex can he detected with DNPH antibodies so as to assess the oxidation level in the tissue, A 5 μl sample of fresh brain tissues was co-incubated with 12% SDS and a 10 μl DNPH working solution at room temperature for 20 min. The sample was neutralized with a 7.5 μl neutralization solution. Samples containing 15 μg total proteins were loaded and detected with Western blot. Overoxidation or oxidation overbalance is an important aspect of the AD pathological mechanism, which facilitates Aβ deposition and formation of neurofibrillary tangles (see, for example, Oxidative Stress and its Implications for Future Treatments and Management of Alzheimer Disease, Int J Biomed Sci, Clark, T. A., et al., 2010, 6(3); p. 225-227).

### 16. Western Blot

The enzymes related with Aβ formation and degradation their molecule levels, the levels of hyperphosphprylated Tau proteins, and the levels of synaptic plasticity correlated proteins were all detected with Western blot. The proteins in the brain tissue homogenates were extracted with a RIP A buffer solution. The samples were transferred to a SDS-PAGE (4-10% acrylamide) gel. The separated proteins were transferred to a nitrocellulose membrane. The blotting was detected with the following antibodies: APP C terminal antibody (171610, Millipore), BACE1 antibody (Millipore), Aβ antibody (6E10, Abcam), Nepriiysin antibody (Millipore), RAGE antibody (Millipore), LRP antibody (5A6, Calbiochem), IDE antibody (epitomics), phosphorylated Tau protein antibody, including pS396 (Signalway), pT231 (Signalway), pS199 (epitomics), pS262 (Abcam), SNAP25 antibody (Millipore), Synaptophysin antibody (Millipore), Synapsin I antibody (Millipore), VAMP1 antibody (Millipore), PSD95 antibody (Millipore), F-actin antibody (Actin) (Sigma-Aldrich). After incubated with IRDye 800CW secondary antibodies, an Odyssey fluorescence scanner is used for scanning.

The results of the present application show that Edaravone has significant intervention effects on multiple key aspects of the Aβ pathologic, process:
(1) Edaravone Inhibits Aβ from aggregating into fibers, and facilitates the Aβ fiber disaggregation

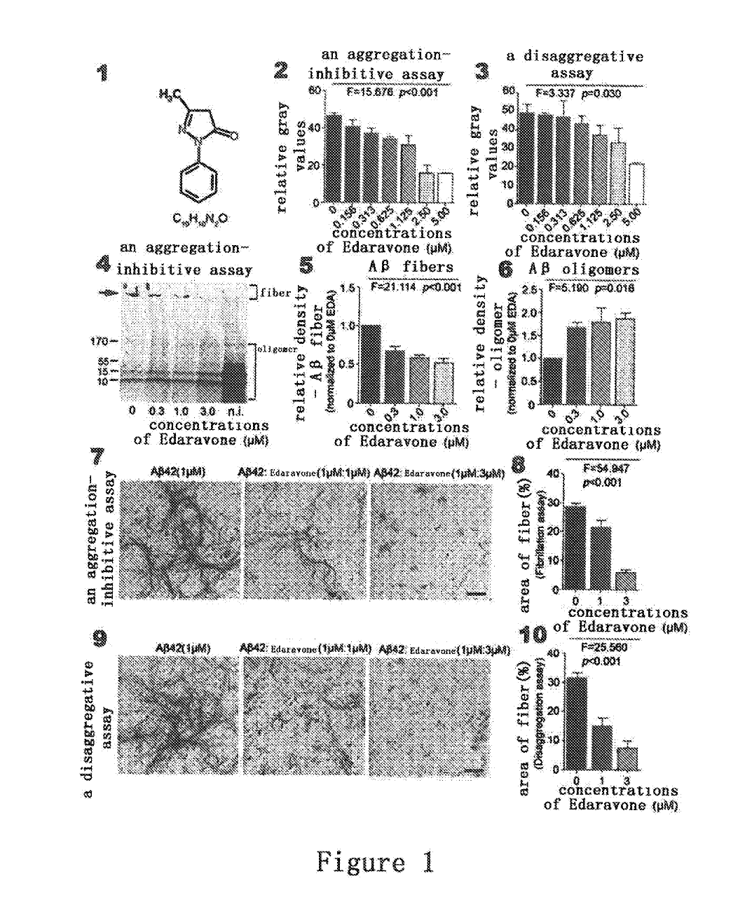

The thioflavin T test demonstrates that Edaravone could effectively inhibit Aβ aggregation in a dose-dependent manner, the higher the concentration of Edaravone, the more significant the effect. At the same time, the disaggregation test demonstrates that Edaravone could also disaggregate the formed Aβ fibers or larger aggregates effectively, the higher the concentration of Edaravone, the more significant this effect (see FIG. 1: 2 and 3). The Western blot further verifies the above conclusion. It is found that 3.0 μM Edaravone could inhibit 50% Aβ from forming aggregates (see FIG. 1: 4-6). The TEM test shows that 3.0 μM Edaravone significantly inhibits Aβ aggregation, Aβ may even unobservable in part of the copper grid. At the same time, the TEM finds that 3.0 μM Edaravone could disaggregate nearly 80% Aβ fibers (see FIG. 1: 7-10).
(2) Edaravone Antagonizes the Cytotoxicity Effect of Aβ

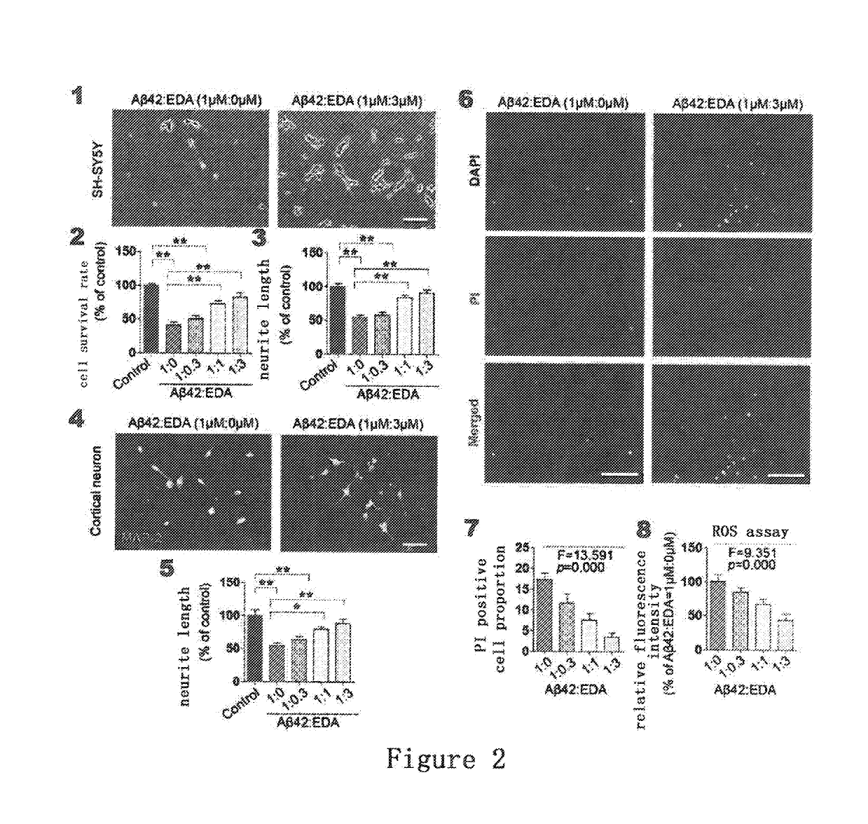

To explore whether Edaravone has an effect of antagonizing Aβ cytotoxicity, SH-SY5Y cell lines and primary cultured cortical neurons were used as the object in the present research, which were added 1 μM Aβ oligomers and treated with different concentrations of Edaravone, and the cell survival rate and the neurite growth were observed. First, it is found in the SH-SY5Y test that 1 μM Aβ oligomers will lead to a substantial cell death (living cells are about 40% of the normal control group) and extensive neurite collapse (neurite length is about 50% of the normal control group). After adding Edaravone, cell survival rate increases significantly, and the cell survival rate increases with the rise of the concentration of Edaravone in a significant dose-dependent manner. At the same time, after adding Edaravone, all-trans-Retinoic acid induced neurite length also increases (see FIG. 2: 1-3). In the primary cultured cortical neuron test, the above effect is also observed (see FIG. 2: 4 and 5), Second, it is found in the ROS test that Edaravone could reverse the Aβ induced oxidative stress (see FIG. 2: 8), In the PI test, Edaravone treatment group has an apoptotic cell ratio significantly lower than in the simple Aβ control group (see FIG. 2: 6 and 7).

### (3) The Application of Edaravone Significantly Improves the Behavioral Performance of the AD Mice

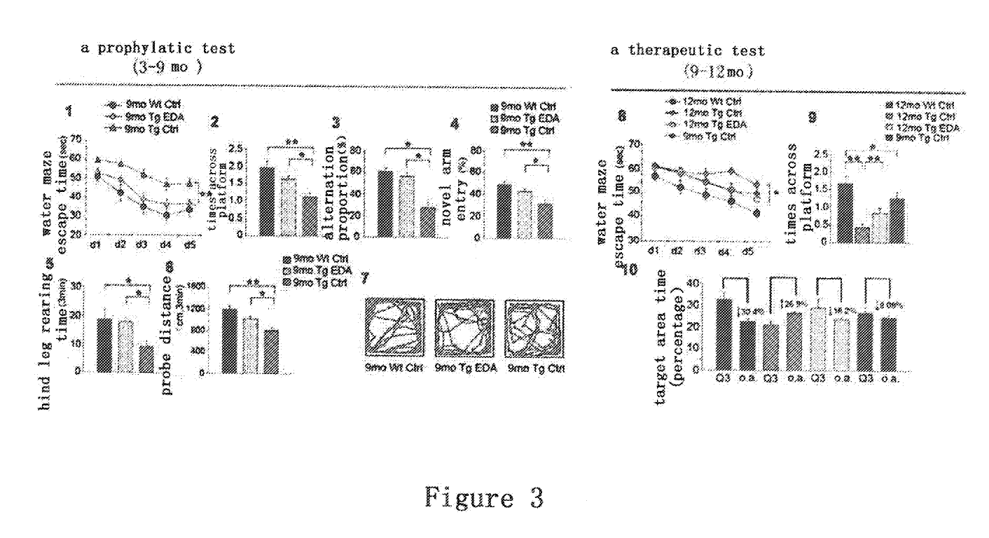

Based on the Morris water maze test, it is found by the present inventors that, no matter in the prophylactic test (see FIG. 3: 1-2) or in the therapeutic test (see FIG. 3: 8-10), behavioral performance of the AD mice in Edaravone treatment group is significantly better than in the control group, presented as the reduction of escape time and the increase in times across platform. It is demonstrated by the Morris water maze that Edaravone could improve the learning and space memory abilities of the AD mice. To comprehensively assess the improvement of the Edaravone on the intelligence of the AD mice, Y-maze, open field test, etc. were also carried out in the present research successively. It is found that, in the spontaneous alternation test and navel arm probe test, the arm entry of the AD mice in the Edaravone treatment group is significantly higher than in the control group, the proportion of the novel arm entry is significantly increased, the alternation proportion is also significantly increased, suggesting that Edaravone could improve the exploring ability and working memory capacity of the AD mice (see FIG. 3: 3 and 4). It is found by the inventors in the open field test that the rearings and the probe distance of the Edaravone treated mice are significantly higher than in the control group (see FIG. 3: 5-7). Similar results are obtained both through ip administration and p.o. administration. It is demonstrated that Edaravone treatments through different routes of administration could improve the intelligence level of the AD mice.

### (4) Edaravone Significantly Reduces the Deposition of Aβ in the Blood Vessels of the Brain

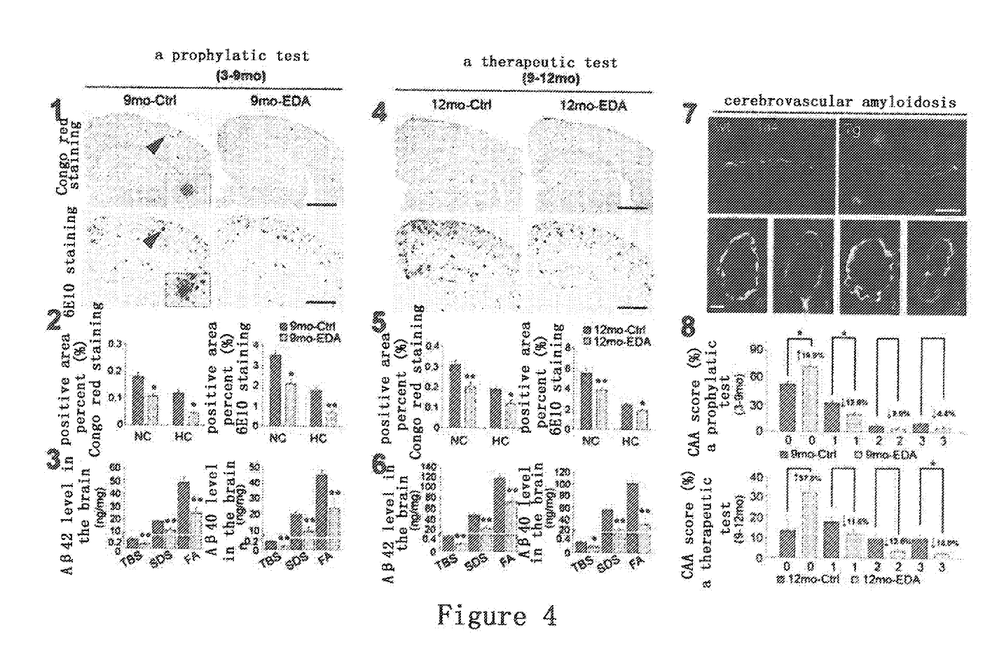

It is found by Congo red staining and 6E10 immunostaining that Edaravone could significantly reduce the Aβ deposition in the brain, being about 50% of the control group (see FIG. 4: 1-2 and 4-5 ). The most valuable is that it is found by double immunofluorescence staining the vascular wall and Aβ, Aβ deposition in the blood vessels of the brain in the Edaravone treatment group is significantly reduced, the proportions of the blood vessels at level 0 and level 1 CAA are significantly increased, the proportions of the blood vessels at level 2 and level 3 are significantly reduced (see FIG. 4: 7 and 8). At the same time, the brain tissues of the AD mice were extracted with TBS, 2% SDS and FA, and extracts of different Aβ aggregation states were obtained, respectively, and the Aβ 40, Aβ 42 levels in the extracts were detected using Covance ELISA kit. It is found that Edaravone could significantly reduce the levels of Aβ at different, aggregation states (see FIG. 4: 3 and 6), which is consistent with the in vitro experimental result. An oral administration also receives the similar result as above.

### (5) Edaravone Reduces the Formation of Aβ by Inhibiting BACE1 Expression

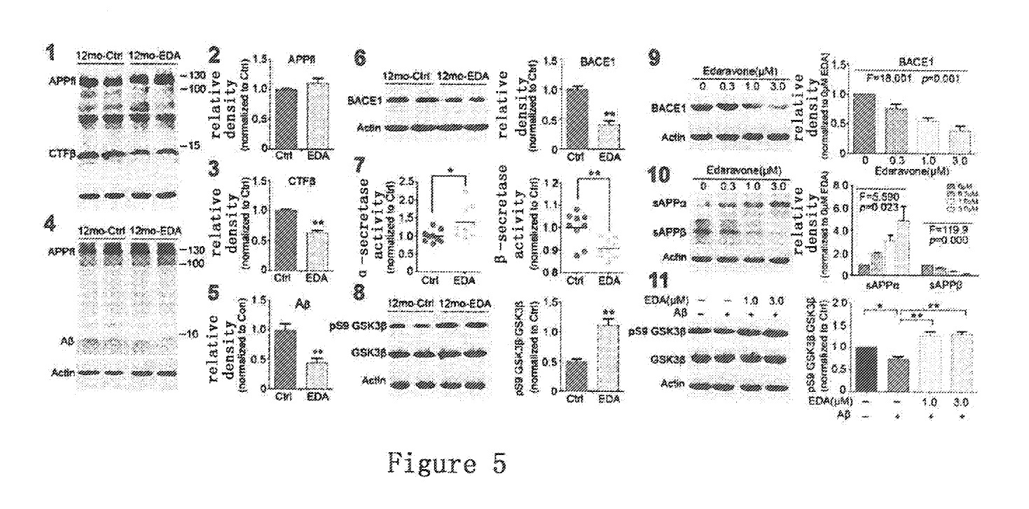

It Is found by Western blot that Edaravone could reduce the formation of Aβ in the brain, decrease the formation of CTFβ (see FIG. 5: 1-5). To explore, the mechanism, it is found that Edaravone has no influence on Aβ metabolizing enzymes such as NEP, IDE, RAGE, LRP, etc., but has a significant influence on α-secretase and β-secretase (BACE1), the cleavage enzymes of APP, presented as the increase of the activity of α-secretase and the significant decrease of the expression and the activity of β-secretase in the Edaravone treatment group (see FIG. 5: 6-7 and 9-10). At the same time, it is also found that Edaravone could inhibit the activity of Gsk-3β (see FIG. 5: 8 and 11).

(6) Edaravone could Reduce the Microglia, Astrocyte and Neural Inflammatory Reactions

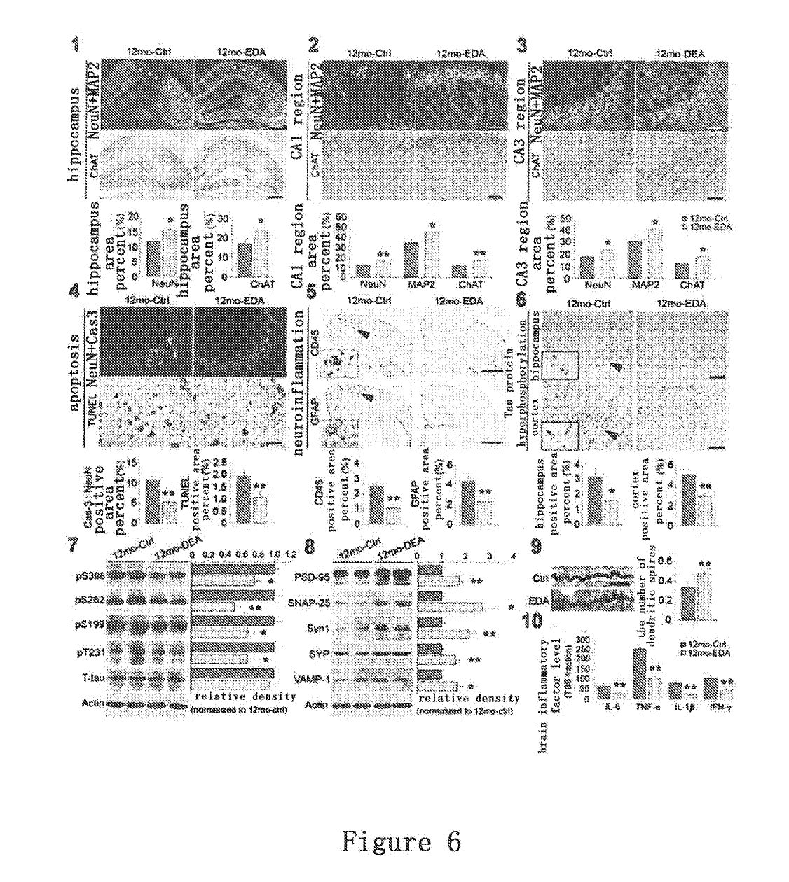

The microglia, astrocyte and neural inflammatory reactions in the brains of AD mice in both prophylactic test and therapeutic test are all reduced significantly. The microglia, astrocyte and neural inflammatory reaction levels in the brains of AD mice in the Edaravone treatment group (p.o.) are significantly reduced, too (see FIG. 6: 5 and 10).

### (7) Edaravone Reduces the Death of Neurons and Synaptic Degeneration

The losses of neurons and the synaptic degeneration are typical clinical manifestations of AD. To explore whether Edaravone has neuron protective effect, immunohistochemistry for MAP-2, NeuN, ChaT etc. was carried out by the inventors. It is found that, as compared with the control group, the NeuN and ChaT positive area ratios are significantly increased in the Edaravone treatment group. It is found by the Golgi staining that the numbers of the dendritic spines are also significantly increased. It is found by Western blot that the expressions of the synapse associated proteins in the Edaravone treatment group are much higher than in the control group (see FIG. 6: 1-3, 4 and 8-9).

### (8) Edaravone Reduces the Phosphorylation Level of Tail Protein

It is found in Western blot that Edaravone could reduce the phosphorylation level of Tau protein at multiple pathological sites (see FIG. 6: 6 and 7). At the same time, it is found in the p396-Tau immunohistochemistry that the p396-Tau positive area ratio is much lower in the Edaravone treatment group.

The descriptions of the above specific embodiments are only for helping understanding the core concept of the present invention. It should be noted that, various improvements and modifications may be made to the technical solutions of the present invention without departing from the principles of the present invention to those skilled in the art, although these improvements and modifications also fall into the range of the claims in the present invention.

1. A method of treating cerebral amyloid angiopathy (CAA) in a patient in need thereof which comprises administering a medicament comprising Edaravone, or an analogue or derivative thereof, to the patient.

2. The method of claim 1, wherein said medicament is administered intravenously, subcutaneously or orally.

3. The method of claim 1, wherein said medicament is formulated into an injection, a tablet or a capsule.

4. The method of claim 1, wherein Edaravone is administered in combination with another CAA therapeutic medicine.

5. The method of claim 4, wherein said other medicine is an Aβ antibody.

6. The method of claim 1, further comprising determining that the patient suffers from CAA before administration.

7. The method of claim 6, wherein the determining refers to determining clinical symptoms of the patient suffering from CAA.

8. The method of claim 1, wherein the patient has characteristic plaques of Alzheimer's disease in the brain.

9. The method of claim 1, wherein the patient has or docs not have symptoms of Alzheimer's disease.

10. The method of claim 1, wherein the patient has experienced a heart attack or stroke.

11. The method of claim 1, wherein a daily dose of Edaravone is between 0.1 mg/kg and 25 mg/kg.

12. A method of treating or preventing CAA in a patient susceptible to CAA which comprises administering a medicament comprising Edaravone, or an analogue or derivative thereof, to the patient.

13. A method of reducing a vascular amyloid protein in a patient in need thereof which comprises administering a medicament comprising Edaravone, or an analogue or derivative thereof, to the patient.

14. The method of claim 13, wherein the administration is carried out according to a therapeutic regime that is related to the removal of the vascular amyloid protein or reduces the incidence of cerebral microbleeds.

15. The method of claim 14, wherein the therapeutic regime comprises monitoring the cerebral microbleeds of the patient by MRI.

16. The method of claim 14, wherein the therapeutic regime comprises monitoring the removal or deposition of the vascular amyloid protein in the blood vessels of the patient by a PET scan.

17. The method of claim 14, wherein the therapeutic regime is a long-term therapeutic regime.

18. The method of claim 15, wherein the therapeutic regime is a long-term therapeutic regime.

19. The method of claim 16, wherein the therapeutic regime is a long-term therapeutic regime.
